(19) United States
(12) Patent Application Publication
(10) Pub. No.: US 2015/0216578 A1
(43) Pub. Date: Aug. 6, 2015

(54) Drill Alignment Device, Method for Manufacturing the Drill Alignment Device and a Method for Removing Bone Cement

(71) Applicants: Bram Lenaerts, Pulle (BE); Kris Govaers, Kalmthout (BE); Jan Demol, Heverlee (BE); Frederik Gelaude, Leuven (BE)

(72) Inventors: Bram Lenaerts, Pulle (BE); Kris Govaers, Kalmthout (BE); Jan Demol, Heverlee (BE); Frederik Gelaude, Leuven (BE)

(73) Assignee: Mobelife N.V., Leuven (BE)

(21) Appl. No.: 14/385,891

(22) PCT Filed: Apr. 4, 2012
(86) PCT No.: PCT/EP2012/056211

§ 371 Date: Dec. 23, 2014

(51) Int. Cl. A61B 17/88; G06F 17/50; G05B 19/4097; A61B 17/17

(52) U.S. Cl. CPC A61B 17/8847; A61B 17/175; G06F 17/50; G05B 19/4097; A61B 17/1631

(57) ABSTRACT

Drill alignment device (6) for use during the removal of bone cement (42) from a cavity (5) resulting from the removal of a cemented prosthetic device (3) from a long bone, such as a femur (2), during the revision of said prosthetic device, wherein the alignment device comprises an elongate body (61) having a size and shape for insertion in the cavity, wherein at least a part of the outer surface of said elongate body is complementary to the corresponding inner surface of said cavity, wherein the alignment device comprises a channel (7) extending through the elongate body between a proximal end (68) and a distal end (64) and wherein said channel is arranged and has a diameter suitable for receiving and guiding a reamer (8) arranged for removing bone cement in a distal part of said cavity

The present invention relates to a drill alignment device, a method for manufacturing a drill alignment device and a method for removing bone cement, i.e. a cement plug, from a distal part of a cavity in a long bone.

In revision surgery to replace a cemented prosthesis in a long bone, for instance the femur, it is of particular importance that the bone cement used to implant the initial prosthesis is removed prior to implanting a new prosthesis. It is known to remove the bone cement from the cavity after extraction of the prosthesis by drilling and reaming.

When removing bone cement from for instance the medullary channel in the femur, care must be taken to minimize the removal or damage to any healthy bone material, as this will negatively influence the rate of success of the revision surgery. In practice it is possible to remove the bone cement from the more proximal parts of the cavity without any guidance using a suitable reamer. Removal of bone cement from the distal end of the cavity, i.e. the cement plug, by reaming is however more critical, as any deviation of the reamer from the central axis in the cavity may result in damage of the bone surrounding the cavity. It is therefore known to use a drill alignment device or drill guide to facilitate drilling or reaming the cement plug.

US 2008/0275566 A1 for instance describes an alignment device comprising an alignment body that comprises a drill guide body and a set of sleeves that support the shaft of the reamers and end mills. This alignment body is attached to the outside surface of the femur by two bone saddles. This device however requires that a large part of the femur has to be exposed during surgery, which increases the complexity of the surgery and subsequent recovery time.

U.S. Pat. No. 4,860,735 describes a similar drill alignment guide.

In U.S. Pat. No. 5,470,336, a cannulated trial femoral component is described having a stem which is shaped substantially the same as the shape of a stem of the original prosthesis. The stem has a longitudinally extending passageway which extends from the distal end to the upper end where it forms an outlet opening. Through this passageway, a hole can be drilled for the insertion of a guidewire in the bone. After removal of the trial femoral component from the bone, the guidewire is then used for guiding reamers having a hollow stem.

Although the above mentioned devices may form suitable means for aligning a reamer, there is still a need for a more efficient drill alignment device which is easy in use.

It is therefore a goal, among other goals, of the present invention to provide an improved, efficient and/or reliable drill alignment device.

This goal, among other goals, is met by a drill alignment device according to appended claims 1.

More in particular, this goal, among other goals, it is met by a drill alignment device for use during the removal of bone cement from a cavity resulting from the removal of a cemented prosthetic device from a long bone, such as a femur, during the revision of said prosthetic device, wherein the alignment device comprises an elongate body having a size and shape for insertion in the cavity, wherein at least a part of the outer surface of said elongate body is complementary to the corresponding inner surface of said cavity, wherein the alignment device comprises a channel extending through the elongate body between a proximal end and a distal end and wherein said channel is arranged and has a diameter suitable for receiving and guiding a reamer arranged for removing bone cement in a distal part of said cavity.

In particular the elongate body, or stem, which is preferably tapered, is arranged to be fitted closely in the cavity in the bone such that its position to the patient's anatomical structure is unique and stable. As the cavity, formed by the remaining bone cement or perhaps surrounding bone, in the long bone has a unique or patient specific shape, forming the elongate body accordingly allows a stable fit.

The outer surface of the elongate body arranged to be in contact with the inner surface of the cavity, is hereto formed complementary to the corresponding section of the inner surface. Complementary in this respect means that the two surfaces in inserted state extend adjacently without any substantial play, or at least with minimal play. The elongate body is most preferably arranged to allow a shape fit in the cavity, such that the elongate body is introducible in the cavity substantially without any wedging forces.

Since the drill alignment device engages the long bone in the cavity left by the removal of the prosthesis, no additional engaging surfaces on the exterior on the bone are needed. No extra exposure of the bone during surgery is therefore needed to align the reamer.

Preferably, the complementary formed outer surface of the elongate body has at least three distinct contact points with corresponding surface areas of the inner surface of the cavity to ensure in a stable fit of the alignment device in the cavity.

The channel has a distal end and a proximal end and is there between preferably surrounded by the body of the alignment device, in particular the elongate body. In other words, the channel is preferably tubular shaped having ends at or near the proximal and the distal ends of the alignment device. It may be advantageously to provide the inner surface of the channel with a friction resistant coating, in case the material of the drill alignment device is not sufficiently friction resistant. Dependent on the used material for manufacturing the alignment device, it may be preferable to provide the walls of the channel with a reinforcing material. In case the used material is relative weak or flexible, the reinforcing material, for instance in the form of a surface or inner wall of the channel, can provide sufficient strength or rigidity to the device.

According to a preferred embodiment of the drill alignment device according to the invention, said channel has a diameter of at least 5.0 mm, preferably at least 6.5 mm or more preferably at least 8.0 mm. Dependent on the reamer to be used, the diameter of the channel is chosen. In this respect, it should be noticed that with a reamer, a tool is meant which is used to create an accurate sized hole. Typical reamers, in the particular the head thereof, used in the removal of bone cement, for instance cement plugs, have a diameter starting from 5 mm.

It should however be noted that also other medical devices can be introduced through to channel, such as endoscopes or ultrasonic tips for providing ultrasonic energy to the cement plug to be removed. In this respect, the invention relates equally well related to an alignment tool for medical devices for use during revision of a prosthetic device.

According to a further preferred embodiment of the drill alignment device according to the invention, substantially the whole outer surface of the elongate body is complementary to the inner surface of said cavity. This in particular allows a stable fit of the drill alignment device in the bone. The elongate body is hereby formed congruent to the inverse of the cavity.

According to a further preferred embodiment at least a part of the outer surface of said elongate body is congruent to the corresponding outer surface of said prosthetic device. As the outer surface of in particular the stem of the prosthetic device corresponds to the surface of the cavity, the contours of the prosthesis may be used as a basis for the design of the drill alignment device as will be explained in more detail below. Preferably, the whole outer surface for contacting the cavity of the elongate body, the part arranged to be inserted in the cavity and arranged for contacting the cavity, is formed congruently to the corresponding surfaces of the prosthesis. In other words, the elongate body and the prosthesis for a large part have the same shape and size. It may for instance be possible that the elongate body has a length smaller than the prosthesis as will be explained below. In any case, the volume of the elongate body will always be enclosed by the volume of the prosthesis to allow the fit of the elongate body in the cavity.

According to a further preferred embodiment of the drill alignment device according to the invention the alignment device preferably comprises a head and is at least partly formed congruently to the prosthetic device. In this embodiment, the drill alignment device is shaped similar to the prosthesis, preferably including the head. In particular the head provides a reference point to the surgeon, as he is then able to position the alignment device until the head of the alignment device reaches substantially the same position as the head of the removed prosthesis. The complimentary or congruent surfaces then facilitate the exact fit of the alignment tool.

It should however be noted that it is not in all cases preferred to design the outer surface of the elongate member based on the outer surface of the prosthesis. For instance, in case of bone resorption around the prosthesis and/or in case the prosthesis is displaced within the bone, the outer surface of the drill guide is preferably designed on the basis of the surrounding bone structures and/or bone cement to allow a stable fit of the alignment device.

The channel device may extend rectilinear with a fixed diameter in the alignment device. However, according to a further preferred embodiment of the drill alignment device according to the invention at least the proximal end of the channel has a curvature towards a medial direction, preferably such that in inserted situation at least a part of the extension of said channel with said curvature does not protrude the greater trochanter. As the extended trajectory of a rectilinear channel due to its diameter would protrude the greater trochanter, the trochanter would be damaged in case a reamer is inserted into this channel. By providing a curvature or bent in at least the proximal end of the channel, at least a part of the extension of said channel does not overlap the trochanter such that upon insertion of a reamer, the trochanter is not damaged. Reamers typically have a shaft showing at least some flexibility, allowing the reamer to conform to the curvature.

The channel may be bent towards for instance the medial plane while the diameter remains unchanged to allow the insertion of the reamer without damaging the neighbouring tissue. It is however also possible that the channel is tapered towards the distal end, i.e. has a decreasing diameter towards the distal end, to the predetermined diameter. The wider proximal end allows easy insertion of the reamer without damaging the tissue, as at least a possible trajectory of the reamer to be introduced does not protrude this surrounding tissue. The narrower distal end then provides sufficient guiding for the reamer such that no bone tissue is damaged while reaming. To increase the guiding properties, preferably at least a length of the channel at the distal end hereto has the predetermined diameter, wherein the channel for instance widens from the end of this length towards the proximal end. In other words, preferably the proximal part of the channel is tapered towards the distal end to the predetermined diameter, wherein more preferably a distal part of the channel has a length having the predetermined diameter.

According to a further preferred embodiment of the drill alignment device according to the invention the elongate body has a length such that the distal end of the elongate body has a diameter corresponding to the sum of the diameter of the channel and a predetermined wall thickness of preferably 3 mm. As the elongate body, or stem, has a tapered shape, wherein the cross sectional area decreases towards the distal end, an end face is chosen which allows said minimal wall thickness. This ensures that the alignment tool, and in particular the elongate body thereof, has sufficient structural integrity. This may thus result in an elongate body having a length shorter than the depth of the cavity or the length of the stem of the prosthesis as already mentioned above.

A further preferred embodiment of the drill alignment device according to the invention is manufactured by a three-dimensional printing technique. Using additive manufacturing techniques, which are known as such, it is possible to manufacture an alignment device with small tolerances from a digital 3d-design. Preferably, the drill alignment device is manufactured from polyamide.

The invention further relates to an assembly of a drill alignment device according to the invention and a reamer. The drill alignment tool is hereby arranged to receive and guide said reamer.

The invention furthermore relates to a method for manufacturing a drill alignment device for use during the removal of bone cement from a cavity resulting from the removal of a cemented prosthetic device from a long bone, such as a femur, during the revision of said prosthetic device, comprising the steps of:
providing surface position data comprising at least positional information of the inner surface of said cavity;
designing an elongate body as a part of the drill alignment device in accordance with the surface position data such that at least a part of the outer surface of said elongate body is complementary to the corresponding inner surface of said cavity;
designing a channel in the drill alignment device extending through the elongate body between a proximal end and a distal end of the alignment device, wherein the channel has a diameter suitable for receiving and guiding a reamer, and;
manufacturing said designed drill alignment device.

As already described above, the drill alignment device is designed to closely fit in the cavity in the bone. The design of the elongate body, the part of the alignment tool arranged for insertion in the cavity, is thereto designed using data comprising the actual geometry of the cavity. The surface position data preferably comprises three-dimensional data being representative of the cavity. The steps of designing preferably comprise designing the alignment device using computer aided design.

According to a preferred embodiment of the method, said channel has a diameter of at least 5.0 mm, preferably at least 6.5 mm or more preferably at least 8.0 mm. This allows the insertion and guidance of correspondingly sized medical devices, in particular a reamer.

According to a preferred embodiment of the method, the surface position data comprises data obtained from a medical scan, for instance a CT-scan, of at least the prosthetic device and the surrounding bone. The surface position date in this case comprises geometrical data of the patient's anatomical structures, including the implanted prosthesis. Based on a the data resulting from a pre-operative scan, for instance a CT-scan, the alignment tool can be manufactured patient specific for allowing a close fit of the device as discussed in detail above.

In this respect it should be noticed that any feature discussed above relating to the drill alignment device as such, is also applicable to the method for designing said device, and vice versa.

It is however also possible that the surface position data data of the outer surface of the prosthetic device comprises. Such data may be obtained from a scan as mentioned above, it is however also possible to derive such data from standard design data of said prosthetic device. Prosthetic devices in general have a generic, i.e. non patient specific, shape. As the cavity in the bone is shaped to this prosthesis, it is possible to shape a corresponding drill alignment device based on the shape of prosthesis to be revised.

According to a further preferred embodiment of method for manufacturing a drill alignment device according to the invention, the step of designing the channel and the elongate body comprises the steps of:
determining a minimal wall thickness of said elongate body;
identifying a cross sectional plane substantially perpendicular to the longitudinal axis of said bone being closest to the distal end of said elongate body which has a surface area suitable for containing the diameter of the channel and the additional minimal wall thickness, and;
designing the elongate body such that said the identified cross sectional plane forms the distal end of the elongate body.

The length of the tapered elongate body of the drill alignment device is according to this embodiment determined by the minimum cross sectional area which is can contain, both in terms of shape and area, both the channel with its predetermined diameter and the additional surrounding predetermined wall thickness. The areas of the cross sections perpendicular to the longitudinal axis of the elongate member, which axis is substantially parallel to the longitudinal axis of the bone and the cavity, decrease in the distal direction due to the tapered shape of the elongate member. Preferably the length of the elongate body is as long as possible, as this increases the stability of the elongate member in the inserted position. Preferably, the step of determining the minimal wall thickness comprises compensating for tolerances in the manufacturing process. Such toleration may include adding 0.6-1.0 mm to the diameters of the channel and the wall thickness.

A further preferred embodiment further comprises the step of providing bone data comprising at least positional data of the surrounding bone and bone cement, wherein the step of designing the channel comprises aligning the channel with the middle of the distal end of the elongate body and the middle of the bone cement in the distal part of the cavity. At least the distal end of the channel is aligned such that the longitudinal axis of the channel extends through both the centre of distal plane or end of the elongate member and the centre of the cement plug.

A further preferred embodiment further comprises the step of providing bone data comprising at least positional data of the surrounding bone, wherein the step of designing the channel comprises designing a curvature in at least the proximal part of the channel towards a medial direction based on said bone data preferably such that an extension of said channel with said curvature does not protrude the greater trochanter. According to this embodiment, at least the proximal end of the channel is designed such that any reamer inserted and guided through the channel will not interfere with the surrounding bone structures, in particular the greater trochanter. It should be noted that any other curvatures in any other direction is also possible to prevent unwanted protrusion of the extended channel through any neighbouring tissue or material.

The curvature may include a bent, wherein the diameter of the channel is substantially the same along the length. It is however also possible, as discussed above, that the channel is tapered towards the distal end. The wider proximal end allows easy insertion of the reamer while preventing damage to surrounding tissue, whereas the narrower distal end having a diameter corresponding to the intended, predetermined diameter, allows safe guiding of the reamer.

According to a further preferred embodiment of method for manufacturing a drill alignment device according to the invention the step of manufacturing the designed drill alignment device comprises three-dimensional printing, preferably from polyamide. As discussed above, this allows an efficient manufacturing process, for instance based on the designs made by computer aided design.

The invention furthermore relates to a method for designing a guide according to the invention, which can be manufactured at a later stage. The invention also relates to a computer readable memory containing data relating to the design of the guide according to the invention.

The invention furthermore relates to a method for removing bone cement from a cavity resulting from the removal of a cemented prosthetic device from a long bone, such as a femur, during the revision of said prosthetic device, comprising the steps of:
introducing a drill alignment device according to the invention in the cavity;
introducing a reamer in the channel of the drill alignment device, and;
operating said reamer until the bone cement in a distal part of said cavity is sufficiently removed.

Using the drill alignment device according to the invention, it is easy an efficient to remove bone cement in distal parts of the long bone, such as the femur.

The present invention is further illustrated by the following Figures, which show a preferred embodiment of the device and method according to the invention, and are not intended to limit the scope of the invention in any way, wherein:

FIG. 1 schematically shows a cross section of a patient with a hip prosthesis through the coronal plane;

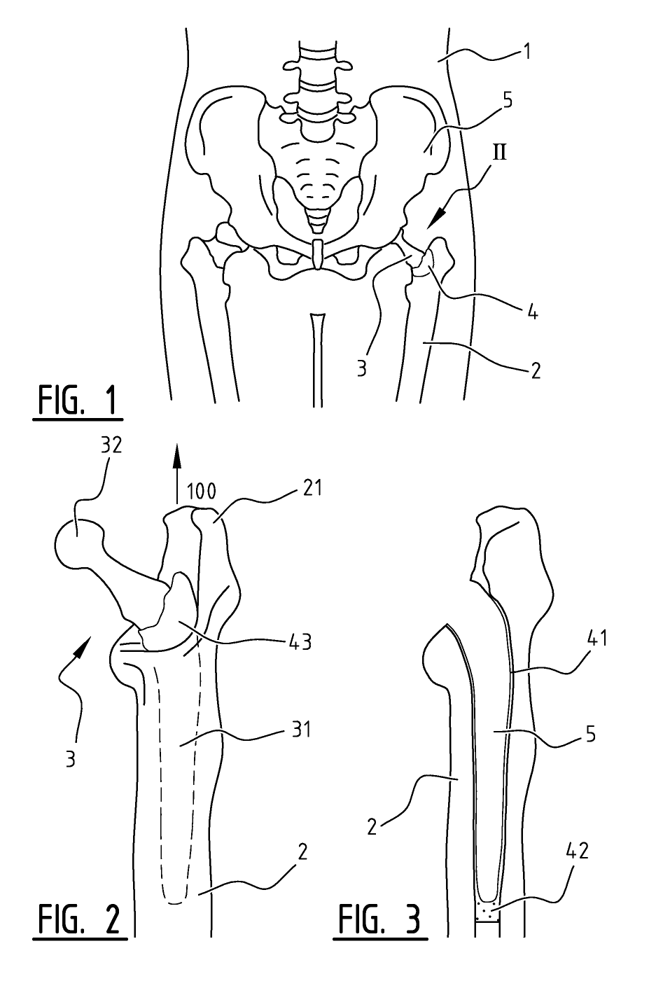

FIG. 2 schematically shows a femur with a prosthesis to be revised in perspective;

FIG. 3 schematically shows the femur of FIG. 2 in cross section along the coronal plane with the prosthesis removed;

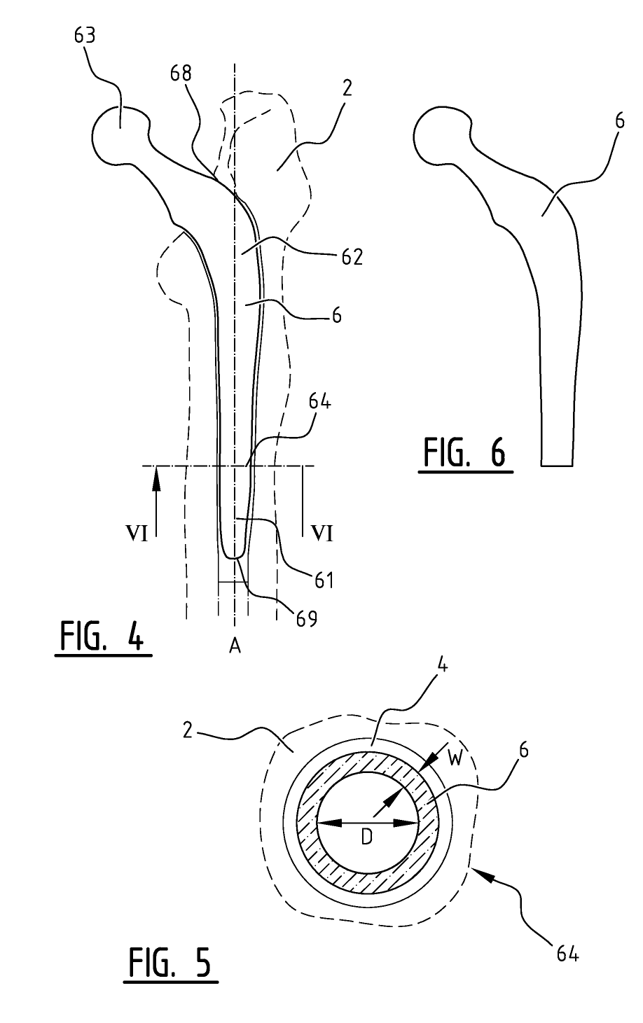

FIGS. 4-6 schematically show the determination of the length of the drill guide;

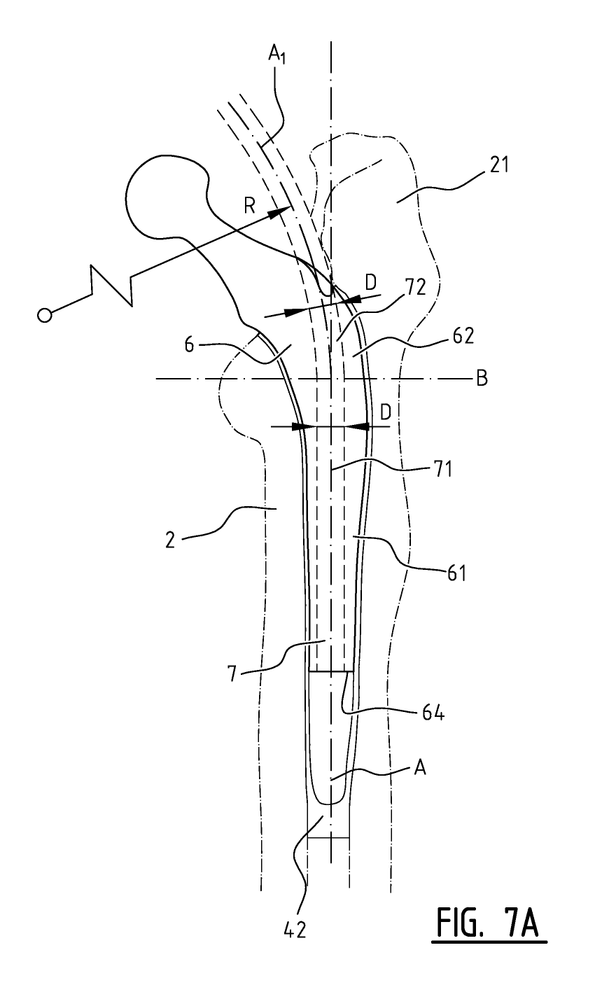

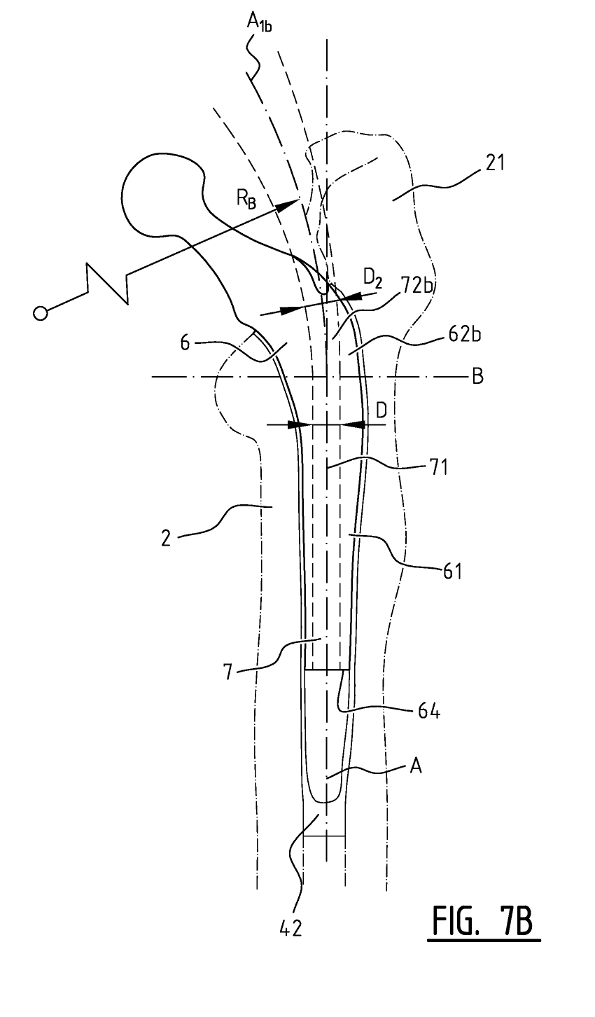

FIG. 7a-b, 8a-c schematically show the design of the channel in the drill guide;

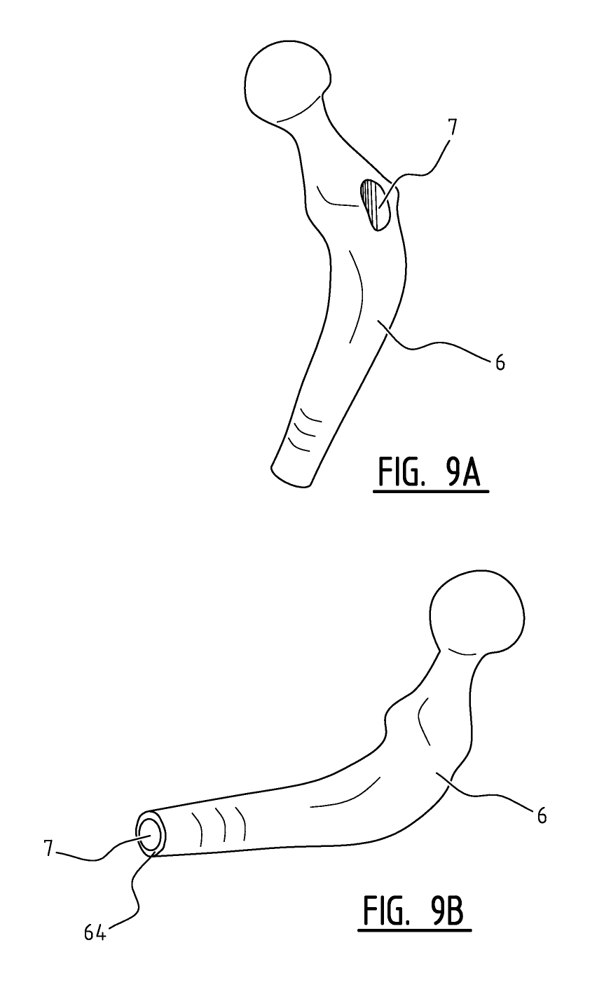

FIGS. 9a and b schematically show the drill guide in perspective from two different views, and;

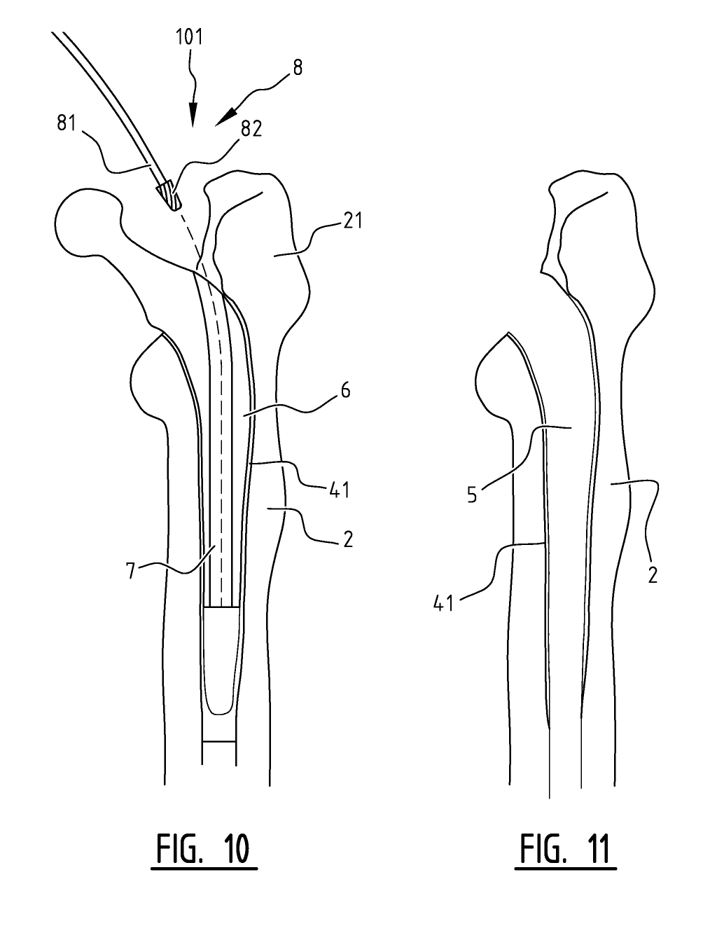

FIGS. 10 and 11 schematically show the femur in cross section in two steps of the method for removing bone cement.

In FIG. 1, a patient 1 in pre-operative state is shown. The left femur 2 is provided with a prosthesis 3 implanted in said femur using bone cement 4. The head 32, see FIG. 2, of the prosthesis 2 engages a corresponding cup (not shows) in the acetabulum of the pelvis 5.

In this example, play between the prosthesis 3 and the femur 2 makes it necessary to replace the prosthesis 3 during revision surgery. After exposing the head of the femur 2 as is known in the art in hip prosthesis revision, any present bone cement 43 at the surface is removed. The stem 31 of the prosthesis 3 is then removed from the bone 2 by pulling the prosthesis, for instance on the head 32, proximally in a direction 100. At the location of the stem 31 of the prosthesis, a cavity 5 is left behind in the femur 2, see FIG. 3.

The cavity 5 is surrounded by bone cement 4 which needs to be removed prior to preparing the femur 2 for the new prosthesis. The bone cement 41 radially surrounding the stem 31 can for instance be removed by scraping or reaming. The cement plug 42 is however more difficult to remove as any tool for removing said plug may damage the surrounding bone in case the tool is off axis. A guide for a tool for the removal of the cement plug is therefore needed.

The guide is designed preoperatively based on a CT scan of the patient. Using image segmentation algorithms and preoperative planning, the shape of the patient's anatomical structures and current prosthetic implants are determined. Therefore, in a first step of designing the guide, the design of the drill guide 6, hereafter also simply the guide, is exactly the same as the shape of the prosthesis 3, such that an exact fit in the bone 2 is assured, see FIG. 4.

The guide 6 is provided with a head 63 corresponding to the size and shape of the head 32 of the prosthesis 3. The head 63 can be used as a reference point for the surgeon during the procedure. The guide 6 is furthermore provided with an elongated body in the form of a stem 61. The stem 61 is arranged for contacting the surfaces of the cavity 5.

In a next step, the length of the stem 61 is determined. To this end, a minimal cross sectional area of the stem 61 is determined based on the intended diameter D of the channel to be created in the guide 6 and a minimal wall thickness W of the guide 6 surrounding said channel, see FIG. 5. Starting from the most distal end 69 of the stem 61 towards the proximal end 68, the first plane 64 is selected having a cross section perpendicular to the longitudinal axis A of the stem 61 which is large enough and is shaped to contain the channel and the surrounding wall. This plane is indicated with the dotted line in FIG. 4. The design of the guide 6, in particular the stem 61, is cut off below this plane, resulting in the design as shown in FIG. 6.

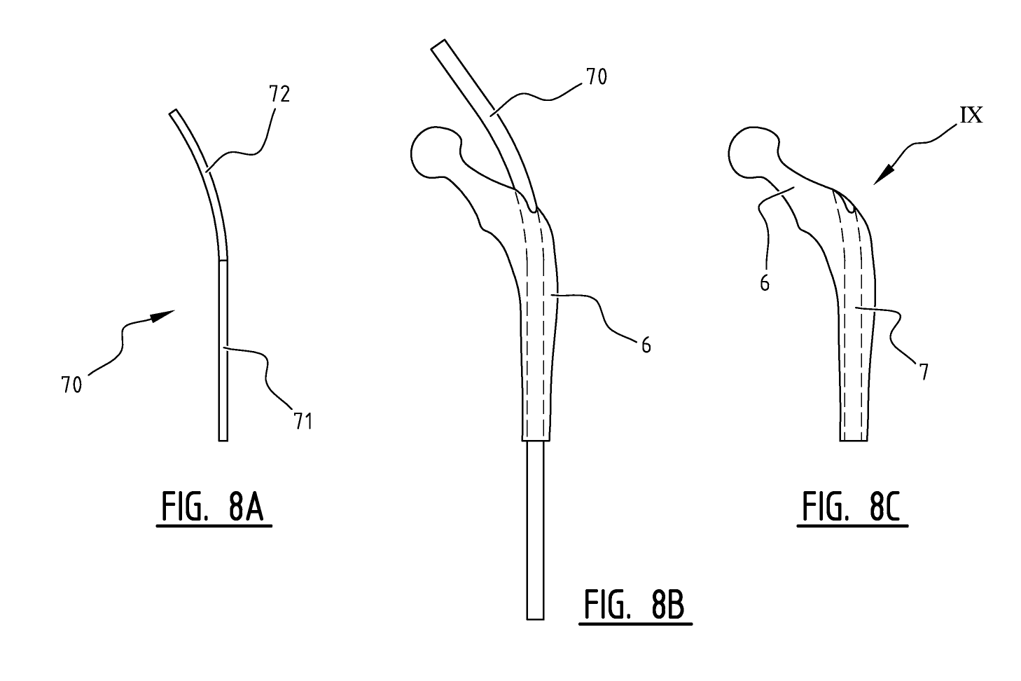

As a next step, the channel 7 for receiving and guiding the channel is designed in the guide 6, see FIG. 7a. To this end, a trajectory 70 of the reamer, see FIG. 8a, is determined which is used to cut out the channel 7 in the design of the guide 6, see FIG. 8b. The trajectory 70, hereafter also simply the channel 7, has a rectilinear distal part 7 and a curved proximal part 72. The distal end 71 of the channel 7 is aligned along the axis A through the centre of the distal end 64 of the guide 6 and the centre of the cement plug 42. Until an upper part 62 of the stem 61, the guide extends rectilinear through said stem 61.

In order to prevent the reamer from damaging bone structures such as the greater trochanter 21, the proximal part 72 of the channel has a curvature with radius R in the medial direction. The radius R is chosen such that the trajectory 70, i.e. the channel and the extension thereof, does not protrude the trochanter 21. At any location the diameter of the channel 7 or the trajectory 70 is at least equal to D.

As an alternative, as shown in FIG. 7b, the proximal part 72b of the channel 7 is tapered towards the distal part 61 of the guide 6. The diameter D2 at the proximal part is therefore greater than the diameter D at the more distal part 71 of the channel 7. Along a length, in this case until line B, from the distal end 64, the channel has a diameter D to ensure proper guiding of the reamer. Next to the tapering, the proximal part 72b is also bent towards the medial plane, see the bent axis indicated with A1b. The wider entrance at the proximal end of the guide allows an easy insertion of the reamer, while at the same time damage to the trochanter 21 is prevented.

Again referring the design of the channel of FIG. 7a, the design of the trajectory 70, see FIG. 8a is subtracted (Boolean) from the design of the of the guide 6, which results in a guide 6 with a channel 7 as shown in cross section in FIG. 7a and in perspective in FIGS. 9a and 9b. The same is of course possible for the channel as shown in FIG. 7b.

The design of the prosthesis 6 made using computer aided design. The next step in the process is to actually manufacture the guide 6 using an additive manufacturing using polyamide. Additive manufacturing or 3d-printing techniques as such are known in the art.

The manufactured guide 6 is used to guide a tool such as a reamer 8 for the removal or drilling of the cement plug 42 in the distal part of the cavity 5, see FIG. 10. To this end, the guide 6 is inserted in the cavity. Due to the design of guide 6, in particular the outer surfaces of the guide 6 being complementary to the surfaces of the cavity 5, in particular the bone cement 41, the guide 6 fits exactly. This allows a stable guide 6 allowing a controlled reaming of the cement plug 42.

The reamer 8, with a head 82 and relatively flexible shaft 81 is inserted into the channel 7 and is extended beyond the distal end of the guide 6 for reaming the cement plug 42. The reamer 8 in this example has a diameter of 8 mm. The reamer 8 is operated until the cement plug 42 is sufficiently removed. During operation of the reamer 8, there is no danger of the reamer 8 being misaligned or being off axis, due to the guidance of the channel 7. This allows controlled reaming of the cement plug as mentioned above.

After reaming, the reamer 8 is withdrawn and also the guide 6 is removed. The bone 2 is then ready for further processing, for instance removing the remaining bone cement 41.

The present invention is not limited to the embodiment shown, but extends also to other embodiments falling within the scope of the appended claims. It is for instance possible to use the drill alignment guide in other long bones than the femur as described, such as the humerus.

1. Drill alignment device for use during the removal of bone cement from a cavity resulting from the removal of a cemented prosthetic device from a long bone, such as a femur, during the revision of said prosthetic device, wherein the alignment device comprises an elongate body having a size and shape for insertion in the cavity, wherein at least a part of the outer surface of said elongate body is complementary to the corresponding inner surface of said cavity, wherein the alignment device comprises a channel extending through the elongate body between a proximal end and a distal end and wherein said channel is arranged and has a diameter suitable for receiving and guiding a reamer arranged for removing bone cement in a distal part of said cavity.

2. Drill alignment device according to claim 1, wherein said channel has a diameter of at least 5.0 mm, preferably at least 6.5 mm or more preferably at least 8.0 mm.

3. Drill alignment device according to claim 1 or 2, wherein substantially the whole outer surface of the elongate body is complementary to the inner surface of said cavity.

4. Drill alignment device according to any of the preceding claims, wherein at least a part of the outer surface of said elongate body is congruent to the corresponding outer surface of said prosthetic device.

5. Drill alignment device according to any of the preceding claims, wherein the alignment device comprises a head and is at least partly formed congruently to the prosthetic device.

6. Drill alignment device according to any of the preceding claims, wherein at least the proximal end of the channel has a curvature towards a medial direction such that in inserted situation an extension of said channel with said curvature does not protrude the greater trochanter.

7. Drill alignment device according to any of the preceding claims, wherein the channel is tapered towards the distal end.

8. Drill alignment device according to any of the preceding claims, wherein the elongate body has a length such that the distal end of the elongate body has a diameter corresponding to the sum of the diameter of the channel and a predetermined wall thickness of preferably 3 mm.

9. Drill alignment device according to any of the preceding claims manufactured by a three-dimensional printing technique, preferably from polyamide.

10. Assembly of a drill alignment device according to any of the preceding claims and a reamer.

11. Method for manufacturing a drill alignment device for use during the removal of bone cement from a cavity resulting from the removal of a cemented prosthetic device from a long bone, such as a femur, during the revision of said prosthetic device, comprising the steps of:
providing surface position data comprising at least positional information of the inner surface of said cavity;
designing an elongate body as a part of the drill alignment device in accordance with the surface position data such that at least a part of the outer surface of said elongate body is complementary to the corresponding inner surface of said cavity;
designing a channel in the drill alignment device extending through the elongate body between a proximal end and a distal end of the alignment device, wherein the channel has a diameter suitable for receiving and guiding a reamer, and;
manufacturing said designed drill alignment device.

12. Method for manufacturing a drill alignment device according to claim 11, wherein said channel has a diameter of at least 5.0 mm, preferably at least 6.5 mm or more preferably at least 8.0 mm.

13. Method for manufacturing a drill alignment device according to claim 11 or 12, wherein the surface position data comprises data obtained from a medical scan, for instance a CT-scan, of at least the prosthetic device and the surrounding bone.

14. Method for manufacturing a drill alignment device according to any of the preceding claims, wherein the surface position data comprises data of the outer surface of the prosthetic device, in particular standard design data of said prosthetic device.

15. Method for manufacturing a drill alignment device according to any of the preceding claims, wherein the step of designing the channel and the elongate body comprises the steps of:
determining a minimal wall thickness of said elongate body;
identifying a cross sectional plane substantially perpendicular to the longitudinal axis of said bone being closest to the distal end of said elongate body which has a surface area suitable for containing the diameter of the channel and the additional minimal wall thickness, and;
designing the elongate body such that said the identified cross sectional plane forms the distal end of the elongate body.

16. Method for manufacturing a drill alignment device according to any of the preceding claims, further comprising the step of providing bone data comprising at least positional data of the surrounding bone and bone cement, wherein the step of designing the channel comprises aligning the channel with the middle of the distal end of the elongate body and the middle of the bone cement in the distal part of the cavity.

17. Method for manufacturing a drill alignment device according to any of the preceding claims, further comprising the step of providing bone data comprising at least positional data of the surrounding bone, wherein the step of designing the channel comprises designing a curvature in at least the proximal part of the channel towards a medial direction based on said bone data such that an extension of said channel with said curvature does not protrude the greater trochanter.

18. Method according to any of the preceding claims, wherein the step of manufacturing the designed drill alignment device comprises three-dimensional printing, preferably from polyamide.

19. Method for removing bone cement from a cavity resulting from the removal of a cemented prosthetic device from a long bone, such as a femur, during the revision of said prosthetic device, comprising the steps of:
introducing a drill alignment device according to any of the preceding claims in the cavity;
introducing a reamer in the channel of the drill alignment device, and;
operating said reamer until the bone cement in a distal part of said cavity is sufficiently removed.
